(19) United States
(12) Patent Application Publication
(10) Pub. No.: US 2026/0139280 A1
(43) Pub. Date: May 21, 2026

(54) Method of making transparent graphene using fermentation

(71) Applicant: ZHI-MIN LI, Taichung City (TW)

(72) Inventor: ZHI-MIN LI, Taichung City (TW)

(21) Appl. No.: 19/452,372

(22) Filed: Jan. 18, 2026

Related U.S. Application Data
(63) Continuation-in-part of application No. 18/140,097, filed on Apr. 27, 2023.

(51) Int. Cl. C12P 3/00

(52) U.S. Cl. CPC C12P 3/00

(57) ABSTRACT

A method of making transparent graphene using fermentation is applied to obtain nano-grade graphene powder by heating raw materials. The powder is mixed with yeast and warm water to cultivate a graphene starter culture. The culture is mixed with edible starch, sugar powder, and warm water and fermented at 38° C. to 40° C. The fermented product is crushed, dried, and pulverized into gray-black powder. A second fermentation is performed, followed by a second drying and pulverizing into white powder. The powder is soaked in warm water at a ratio of 1:500, and an upper layer of the warm water is removed.

### BACKGROUND OF THE INVENTION

This application is a Continuation-in-Part of application Ser. No. 18/140,097, filed Apr. 27, 2023.

### FIELD OF THE INVENTION

The present invention relates to a method of making transparent graphene using fermentation, and more particularly to the method for producing transparent graphene containing probiotics through fermentation.

### DESCRIPTION OF THE PRIOR ART

Graphene is a two-dimensional monolayer crystal composed of carbon atoms arranged in a honeycomb crystal lattice structure formed by sp² hybrid orbitals. The structure of graphene is highly stable, and this stable lattice configuration endows graphene with excellent thermal conductivity. Scientists have discovered that graphene oxide is highly effective in inhibiting the growth of Escherichia coli (E. coli) without causing harm to human cells. Graphene oxide also exhibits antibacterial properties against other types of bacteria. Derivatives of graphene are considered to be potentially applicable in cancer treatment. The underlying principle is that graphene oxide can distinguish between the electronic density of cancer cells and normal cells, allowing it to attach to cancer stem cells. This attachment enables targeted drugs to act on the cells and suppress the distant metastasis of tumors. This breakthrough discovery compensates for the limitations of traditional chemotherapy and radiotherapy, which can only eliminate differentiated cancer cells, and is expected to result in higher treatment response rates and patient survival rates. The research team has demonstrated through experiments that graphene oxide can inhibit the formation of tumor spheres in six types of cancer (breast, pancreatic, brain, lung, ovarian, and prostate), thereby suppressing their spread.

The present invention has arisen to mitigate and/or obviate the afore-described disadvantages.

### SUMMARY

A primary aspect of the present invention is to provide a method of making transparent graphene using fermentation by which the transparent graphene-based product containing probiotics is produced. The product not only does not harm human cells, but also exhibits antibacterial properties, thus achieving the purpose of improving and alleviating discomfort symptoms in the human body.

To obtain above-mentioned aspect, a a method of making transparent graphene using fermentation provided by the present invention contains steps of:
pre-processing, wherein raw materials for producing graphene, Moso bamboo, are first heated to 800° C., and then further heated to 1000° C. to form nano-grade graphene powder; the nano-grade graphene powder is then mixed with yeast and added into warm water, and the mixture is placed in a temperature-controlled room for two days to cultivate a graphene starter culture;
1) Mixing and making first fermentation, wherein the graphene starter culture, edible starch, and sugar powder are poured into a container; warm water is then added, stirred evenly, and the mixture is placed in the temperature-controlled room at 38° C. to 40° C. for fermentation for three days to generate graphene enzyme powder;
2) Crushing and drying, wherein the graphene enzyme powder is mashed and evenly spread on a stainless steel platform, and then placed in the temperature-controlled room to dry for three to four days;
3) Pulverizing, wherein the dried graphene enzyme powder is placed into a powder mixer and pulverized into gray-black powder;
4) Making second fermentation, wherein the gray-black graphene enzyme powder is again mixed with the graphene starter culture, edible starch, and sugar powder, and the mixture is poured into a container; warm water is then added, stirred evenly, and the mixture is placed in the temperature-controlled room at 38° C. to 40° C. for fermentation for three days;
5) Drying after the second fermentation, wherein the graphene enzyme powder is mashed and evenly spread on a stainless steel platform, and then placed in the temperature-controlled room to dry for three to four days;
6) Making a final pulverization, wherein the dried graphene enzyme powder is placed into a powder mixer and pulverized into white powder, thus completing the second fermentation;
7) Soaking and separating, wherein the graphene enzyme powder obtained after the second fermentation is mixed with warm water at a ratio of 1:500, soaked for one day, after which the edible starch naturally precipitates and aggregates at the bottom, and an upper layer of water above the precipitated edible starch is removed; and
8) Producing a finished product, wherein a graphene-based product is obtained, which is transparent and contains probiotics.

The raw material used in the pre-processing step for producing the graphene is Phyllostachys edulis (Moso bamboo).

The cultivation time of the graphene starter culture in the pre-processing step is two days.

The temperature in the temperature-controlled room in step 1) and step 4 is raised to 38° C. to 40° C.

The fermentation time in step 1) is three days.

The container used in step 1) and step 4) is selected from one of a plastic bucket or a glass jar.

The platform used in step 2) and step 5) is made of stainless steel.

The drying time in step 2) and step 5) is three to four days.

The graphene enzyme powder in step 3) is gray-black in color.

The graphene enzyme powder in step 6 is white in color.

The ratio of the graphene enzyme powder to the warm water in step 7 is 1:500.

### BRIEF DESCRIPTION OF THE DRAWINGS

FIG. 1 is a block diagram showing steps of a method of making transparent graphene using fermentation according to a preferred embodiment of the present invention.

FIG. 2 is a block diagram showing the ratio of maxing the graphene fermentation powder according to the preferred embodiment of the present invention.

FIG. 3 is a schematic diagram showing the operation of soaking and stirring the graphene enzyme powder in warm water, allowing the edible starch to naturally precipitate and aggregate at the bottom, and removing an upper layer of the warm water containing transparent graphene with probiotics.

### DETAILED DESCRIPTION

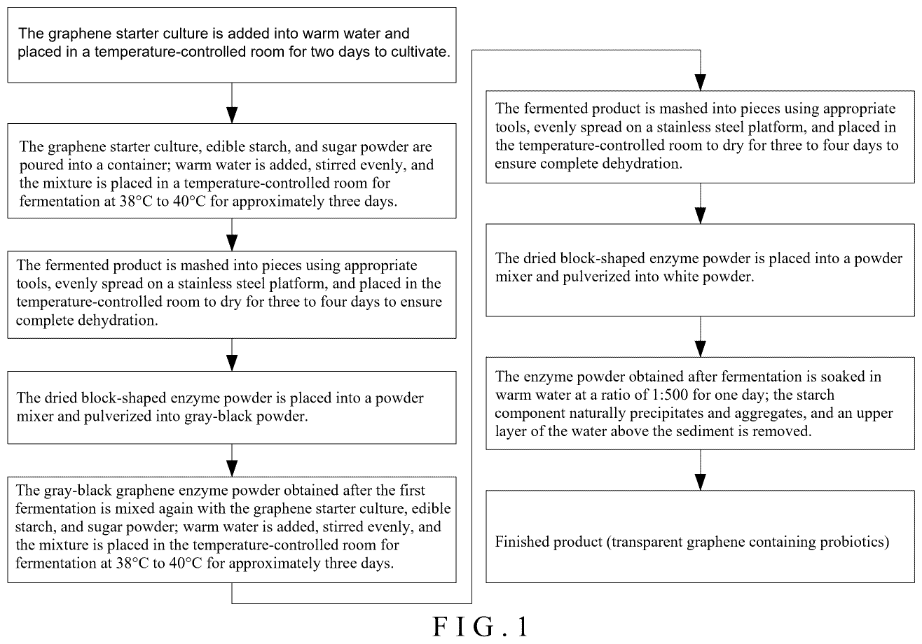

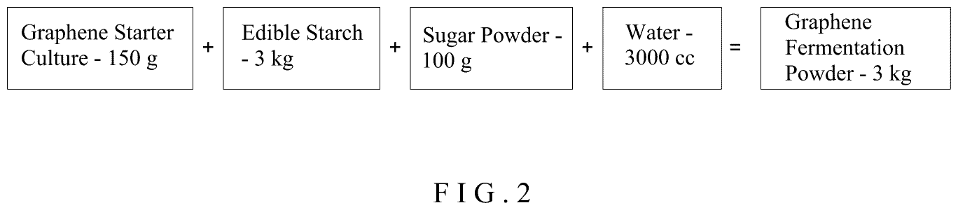

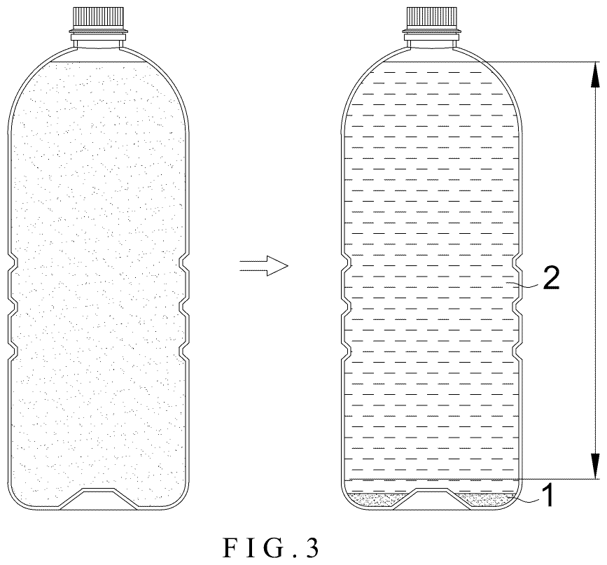

FIG. 1 is a block diagram showing steps of a method of making transparent graphene using fermentation according to a preferred embodiment of the present invention. The method comprises steps of:
pre-processing, wherein raw materials for producing graphene (e.g., Phyllostachys edulis, commonly known as Moso bamboo) are first subjected to a first heating process at 800° C., followed by a second heating process at 1000° C.; after cooling, the material becomes nano-grade graphene powder, the nano-grade graphene powder is then mixed with yeast and added into warm water, and a mixture is placed in a greenhouse or temperature-controlled room for two days to cultivate a graphene starter culture;
1) Mixing and making first fermentation, wherein the graphene starter culture is mixed with edible starch (e.g., sweet potato starch, corn starch), sugar powder, and warm water at 30° C. (as shown in FIG. 2; the ratio is: 150 g of the graphene starter culture, 3 kg of edible starch, 100 g of sugar powder, and 3000 cc of warm water) to form 3 kg of a graphene fermentation powder; the mixture is poured into a food-grade container (e.g., a plastic bucket, a glass jar), stirred evenly, and placed in the temperature-controlled room at 38° C. to 40° C. for fermentation for approximately three days;
2) Crushing and drying, wherein a fermented product is mashed by using appropriate tools and evenly spread on a stainless steel platform, then placed in the temperature-controlled room to dry for three to four days to ensure complete dehydration;
3) Pulverizing, wherein a dried, block-shaped graphene enzyme powder is placed into a powder mixer for pulverization; a pulverized powder is then placed into a food-grade container (e.g., a plastic bucket or a glass jar), and a pulverized graphene enzyme powder is gray-black in color;
4) making second fermentation, wherein a gray-black graphene enzyme powder obtained after the first fermentation is mixed again with a graphene starter culture, edible starch, and sugar powder, and the mixture is poured into a container; warm water is then added, stirred evenly, and the mixture is placed in the temperature-controlled room for fermentation at 38° C. to 40° C. for three days;
5) Drying after the second fermentation, wherein a graphene enzyme powder obtained from the second fermentation is mashed and evenly spread on the stainless steel platform, and then placed in the temperature-controlled room to dry for three to four days;
6) Making a final pulverization, wherein the graphene enzyme powder that has been completely dried is placed into the powder mixer and pulverized into a white powder, thus completing the second fermentation;
8) Soaking and separating, wherein the graphene enzyme powder obtained after the second fermentation is soaked in warm water at a ratio of 1:500 for one day; after an edible starch component naturally precipitates and aggregates, an upper layer of the warm water is removed (as shown in FIG. 3); and
7) Producing a finished product, wherein a graphene-based product (designated as Product 2) is obtained, which is transparent and contains probiotics, thus producing the finished product.

Thereby, the transparent graphene-based product (i.e. Product 2) containing probiotics is produced. The product not only does not harm human cells, but also exhibits antibacterial properties, thus achieving the purpose of improving and alleviating discomfort symptoms in the human body.

When introducing elements of the present invention or the preferred embodiments thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above constructions, products, and methods without departing from the scope of the invention, it is intended that all matter contained in the above description and shown in the accompanying drawings shall be interpreted as illustrative and not in a limiting sense.

What is claimed is:

1. A method of making transparent graphene using fermentation, comprising steps of:
pre-processing, wherein raw materials for producing graphene, Moso bamboo, are first heated to 800° C., and then further heated to 1000° C. to form nano-grade graphene powder; the nano-grade graphene powder is then mixed with yeast and added into warm water, and the mixture is placed in a temperature-controlled room for two days to cultivate a graphene starter culture;
1) Mixing and making first fermentation, wherein the graphene starter culture, edible starch, and sugar powder are poured into a container; warm water is then added, stirred evenly, and the mixture is placed in the temperature-controlled room at 38° C. to 40° C. for fermentation for three days to generate graphene enzyme powder;
2) Crushing and drying, wherein the graphene enzyme powder is mashed and evenly spread on a stainless steel platform, and then placed in the temperature-controlled room to dry for three to four days;
3) Pulverizing, wherein the dried graphene enzyme powder is placed into a powder mixer and pulverized into gray-black powder;
4) Making second fermentation, wherein the gray-black graphene enzyme powder is again mixed with the graphene starter culture, edible starch, and sugar powder, and the mixture is poured into a container; warm water is then added, stirred evenly, and the mixture is placed in the temperature-controlled room at 38° C. to 40° C. for fermentation for three days;
5) Drying after the second fermentation, wherein the graphene enzyme powder is mashed and evenly spread on a stainless steel platform, and then placed in the temperature-controlled room to dry for three to four days;
6) Making a final pulverization, wherein the dried graphene enzyme powder is placed into a powder mixer and pulverized into white powder, thus completing the second fermentation;
7) Soaking and separating, wherein the graphene enzyme powder obtained after the second fermentation is mixed with warm water at a ratio of 1:500, soaked for one day, after which the edible starch naturally precipitates and aggregates at the bottom, and an upper layer of water above the precipitated edible starch is removed; and
8) Producing a finished product, wherein a graphene-based product is obtained, which is transparent and contains probiotics.
